(19) United States
(12) Patent Application Publication
(10) Pub. No.: US 2021/0128860 A1
(43) Pub. Date: May 6, 2021

(54) ENDOTRACHEAL TUBE FIXATION

(71) Applicant: Novo Klinik Service GMBH, Bergheim (DE)

(72) Inventors: Herman VAN DER VEGT, Utrecht (NL); Wilfred TEUNISSEN, Utrecht (NL); Freerk VAN OUDHEUSDEN, Voorhout (NL)

(73) Assignee: Novo Klinik Service GMBH, Bergheim (DE)

(21) Appl. No.: 17/056,930

(22) PCT Filed: May 21, 2019
(86) PCT No.: PCT/EP2019/063154

§ 371 Date: Nov. 19, 2020

(30) Foreign Application Priority Data
May 22, 2018 (EP) 18173703.2

(51) Int. Cl. A61M 16/04

(52) U.S. Cl. CPC A61M 16/0497; A61M 16/0493

(57) ABSTRACT

A fixation device (1) for a medical line (L) to a patient is provided. The medical line may be a wire, a tube, an endotracheal tube or similar. The fixation device comprises a support member (3) supporting a retainer (5), the retainer defining an axis and being adapted to retain the medical line extending in axial direction. The retainer comprises an enclosure (25) for radially retaining the line and a clamp (27). The clamp is configured to provide a crimping force around the axis for fixing the line in at least axial direction, preferably also radial direction, relative to the enclosure.

### TECHNICAL FIELD

The present disclosure relates to the field of fixing an intubation line or other line to a patient, in particular to the field of endotracheal tube fixation devices.

### BACKGROUND

Patients that are incapable of breathing may be ventilated via intubation, i.e. placement of an endotracheal tube into the throat of the patient. E.g. for proper ventilation of the patient and/or for proper control of the ventilation and/or for prevention of harm to the patient by the inserted foreign object the position of the tube must be reliably fixed to the patient, both in longitudinal direction of the tube and in radial direction thereto. Similar considerations apply for other lines introduced into a patient, e.g. feeding lines, sensor probes, catheters, drains, etc.

Medicinal lines may be in use for prolonged periods of care and patient-friendliness is therefore an important consideration. The line must be exchangeable for adaptation, for cleaning of the patient and/or the line, for adjustment of treatment, for administration of medicaments and/or food etc., and similar instances. User friendliness for care givers is therefore important as well.

Moreover, various types and/or sizes of tubes may be used for different treatments. Further, intubation must sometimes be applied under significant pressure and haste, e.g. in case of an accident and/or in case of short staffing.

The various operating conditions that may be encountered therefore have led to commercially available fixation device optimised for different uses.

An exemplary securement system for an endotracheal tube is disclosed in WO 2010/033109 A1: a securement system securing an endotracheal tube or other medical article in position upon a patient and arresting movement of the endotracheal tube. The securement system includes a support member and a retainer. The retainer defines a channel configured to receive the endotracheal tube. A head securement member attaches the support member to the head of the patient. The securement system can include a soft gel for placement against the patient's skin and a track for securing the endotracheal tube at a plurality of locations relative to the support member. The securement system can maintain an endotracheal tube in position upon a patient and allow access to the patient's oral cavity.

Herewith, further improvements to endotracheal tube fixation are provided.

### SUMMARY

A fixation device for a medical line to a patient is provided. The medical line may be a wire, a tube or similar. In particular the medical line may comprise an endotracheal tube and/or a gastric tube and/or the line may be associated with a laryngeal mask, e.g. as an integral whole. The fixation device comprises a support member supporting a retainer, the retainer defining an axis and being adapted to retain the medical line extending in axial direction. The retainer comprises an enclosure for radially retaining the line and a clamp. The clamp is configured to provide a crimping force around the axis for fixing the line in at least axial direction, preferably also radial direction, relative to the enclosure. Herein, terms like axial and radial relate to the axis.

The crimping force is a centripetal force in a net radial direction towards the axis, e.g. a summation of plural localised radial force contributions distributed over more than 180 degrees around the axis.

Thus, the fixation device can provide radial retainment and axial fixation, preferably also radial fixation. This facilitates initial placement of the line, possibly adjustment of the line to a desired position and/or orientation relative to the patient and/or to the enclosure, and fixation of the line by the clamp. The device can clamp the line without localised or one-sided pinching force, e.g. preventing risks of closing or damaging a tube e.g. by squeezing and/or buckling.

The clamp may be configured to provide a substantially axisymmetric clamping force distribution. This may center the line and facilitate positioning it.

The clamp may comprise a deformable member which may be configured to be tightened in circumferential direction around the axis. The deformable member may comprise one or more of a flexible strap, a belt and a chain. This may facilitate conforming the clamp to a shape of the line, e.g. in case the line comprises two line parts with different sizes adjacent each other such as a multi-bore tube with a small-bore tube attached to a larger-bore tube. Tightening in circumferential direction may distribute clamping forces about the line.

The clamp may comprise one or more radial and inward protrusions, which may provide locally increased clamping force and/or provide increased friction, either or both increasing reliability in establishing and/or maintaining a position and/or orientation of the line, e.g. relative to at least the enclosure.

The clamp may be adjustable to accommodate and clamp different shapes and/or sizes of lines, e.g. lines of different diameter, e.g. to provide a suitable crimping force around the axis fixing the respective line. The adjustability may be one-directional, e.g. only allowing size reduction, crimping and/or increasing force, or be reversible, e.g. allowing also relaxation and/or size increase.

The clamp may comprise one or more indexing features and/or fixation features for establishing a size and/or a clamping force. In particular, the clamp may comprise indexing features and/or fixation features for defining one or more predetermined relative positions of clamp portions, such as one or more latches and/or ratchets, for establishing different clamp sizes and/or clamping forces. An indexing feature, in particular a latch may facilitate a snap lock for closing the clamp relative to the axis, e.g. encircling the axis.

The clamp may be openable and closable, in an open state accepting a line to be clamped in another direction than axial direction, e.g. sideways in a radial direction, and in a closed state configured for clamping the line. The opening and/or closing of the clamp may be reversible, e.g. facilitating exchanging the line.

The enclosure and/or the clamp may extend over more than 180 degrees around the axis, and preferably fully surrounding the axis.

The enclosure may extend over more than 180 degrees around the axis in an axial position, e.g. in one radial plane and preferably it may fully surround the axis in an axial position, e.g. in one radial plane. This may prevent step edges in axial direction of support structures for the line and/or it may prevent abrupt deviations in forces on the tube or line, which might cause localised pinching, bending and/or damaging of the line.

The retainer may be openable and closable, in an open state accepting a line to be fixed in another direction than an axial direction, e.g. sideways, in a radial direction, and in a closed state being configured for retaining and clamping the line by the enclosure and clamp respectively. The opening and/or closing of the retainer may be reversible, e.g. facilitating exchanging the line. In particular, the enclosure may be openable and closable, in an open state accepting a line to be retained in another direction than an axial direction, e.g. sideways in a radial direction, and in a closed state configured for radially retaining the line. The opening and/or closing of the enclosure may be reversible, e.g. facilitating exchanging the line. Also, the clamp may be openable and closable, in an open state accepting a line to be clamped in another direction than axial direction, e.g. sideways in a radial direction, and in a closed state configured for clamping the line. The opening and/or closing of the clamp may be reversible, e.g. facilitating exchanging the line.

The enclosure may comprise plural parts hingedly connected for opening and closing the enclosure, e.g. two or more C-shaped parts, the hinging axis may extend in axial direction. Such C-shaped parts may extend over more or less than 180 degrees around the axis, by equal or mutually different amounts. This may provide a large opening and/or otherwise facilitate arranging a line into the closure. The hinge may be arranged offset from the axis. E.g., two or more C-shaped parts may be hingedly connected at adjacent respective tips, enabling moving the parts from a combined C- or O-shape for radially retaining the endotracheal tube, to a more-or-less U- or W-shape for receiving the line. The opening and/or closing of the enclosure may be reversible, e.g. facilitating exchanging the line.

The enclosure may comprise a snap lock for closing. This may facilitate single-handed closing the closure. A snap lock may be particularly effective for closing an enclosure comprising hingedly connected C-shaped parts. A snap lock may be openable, for which a release tab may be provided. Also, the clamp may comprise a snap lock for closing.

The clamp may be connected to plural parts of the enclosure and be openable and closable together with the enclosure. This may facilitate arranging a tube or line into the closure and/or into the clamp.

The clamp may comprise a clamp member, which may be deformable, which is attachable or attached to two enclosure parts that are movable with respect to each other for opening and/or closing the enclosure. Thus, the clamp may be deformed, in particular opened (further) or closed (further) in conjunction with deformation, in particular opening (further) or closing (further) of the enclosure.

The clamp may be accommodated at least partly within the enclosure. In particular, the clamp may be accommodated in the enclosure in radial and/or in axial direction. The enclosure may cover the clamp in axial and/or radial direction. This may protect at least part of the clamp. Also, this may facilitate operating the closure and the clamp, e.g. one or more of (re-)opening, (re-)closing and (re-)adjusting the clamp, if and where applicable.

The retainer may be movable and adjustable relative to the support member to plural positions on the support member, in particular in at least a lateral direction with respect to a proximal and distal direction. The proximal and distal direction may be associated with the axial direction of the retainer.

The support member may comprise a lateral track and the retainer may be movable, in particular translatable along the track.

The support member and the retainer may comprise cooperating indexing features and/or fixation features for defining and/or fixing one or more relative positions and/or orientations. In particular, the support member and the retainer may comprise one or more latches and/or ratchets. Adjustment of such relative positions and/or orientations may be independent on the retention and/or clamping of the line in the retainer.

The support member may be configured for head-mounting of the fixation device, comprising one or more facial rest portions, e.g. cheek rests, and attachment portions for one or more head bands.

The support member may comprise a bridge for maintaining one or more portions of the support member free from skin contact. The bridge may comprise or support the lateral track.

One or more of the facial rest portions may be provided with a skin contact pad, which may be adhesive and/or detachably attached to the respective facial rest portions, e.g. for replacement. For detachable attachment the support member may comprise cooperating fixation parts, e.g. a clamp.

The fixation device may be provided as an assembly together with a medical line such as a wire, a tube which may be an endotracheal tube, or similar. The assembly may be packaged and/or be sterile.

### BRIEF DESCRIPTION OF THE DRAWINGS

The above-described aspects will hereafter be more explained with further details and benefits with reference to the drawings showing a number of embodiments by way of example.

FIG. 1 shows a fixation device in perspective view;

FIG. 2 is an exploded view of the device of FIG. 1;

FIGS. 3-5, 6-8, and 9 show the retainer of the device of FIG. 1 in different perspective views, exploded views, and a partial cross section view, respectively;

FIGS. 10-14 show different stages of use of the device;

FIG. 15 shows the device in use on a person's head.

### DETAILED DESCRIPTION OF EMBODIMENTS

It is noted that the drawings are schematic, not necessarily to scale and that details that are not required for understanding the present invention may have been omitted. The terms “upward”, “downward”, “below”, “above”, and the like relate to the embodiments as oriented in the drawings, unless otherwise specified. Further, elements that are at least substantially identical or that perform an at least substantially identical function are denoted by the same numeral, where helpful individualised with alphabetic suffixes.

Further, unless otherwise specified, terms like “detachable” and “removably connected” are intended to mean that respective parts may be disconnected essentially without destruction of either part, e.g. excluding structures in which the parts are integral (e.g. welded or molded as one piece), but including structures in which parts are attached by or as mated connectors, fasteners, releasable self-fastening features, etc.

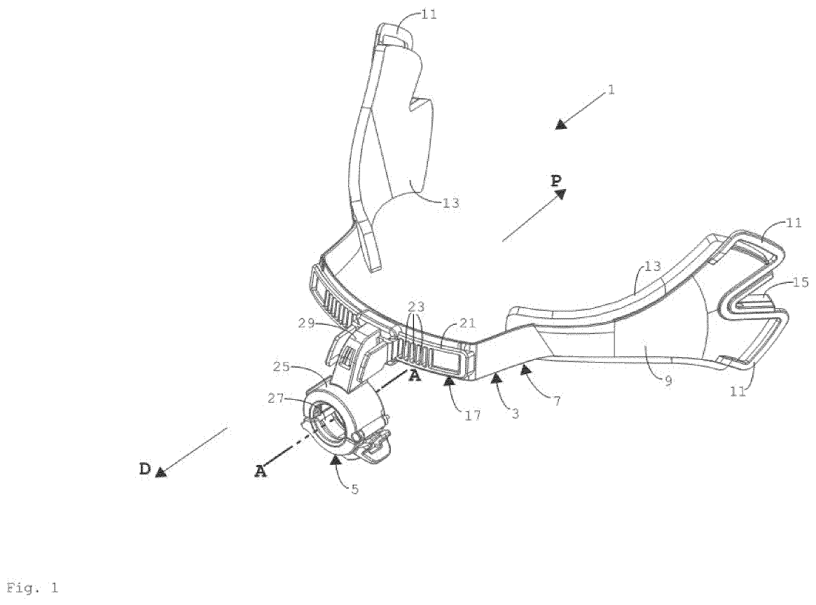

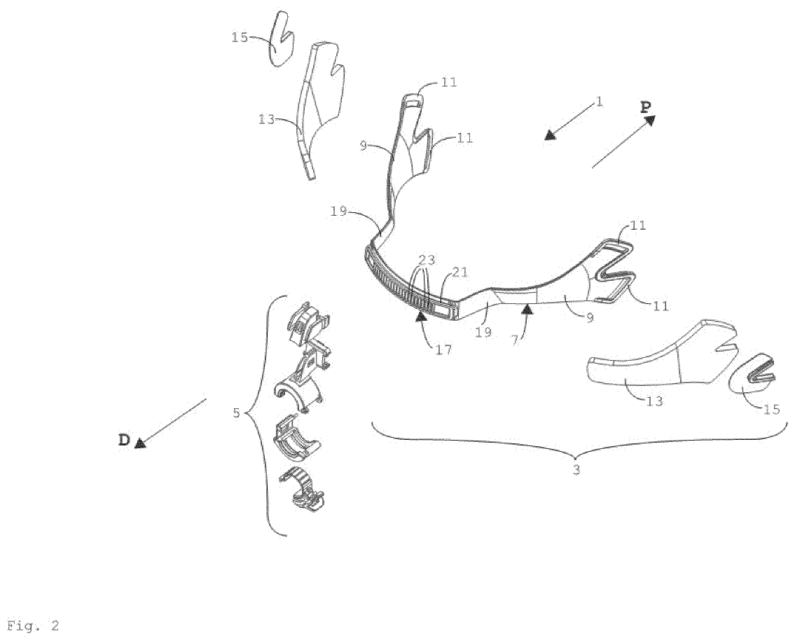

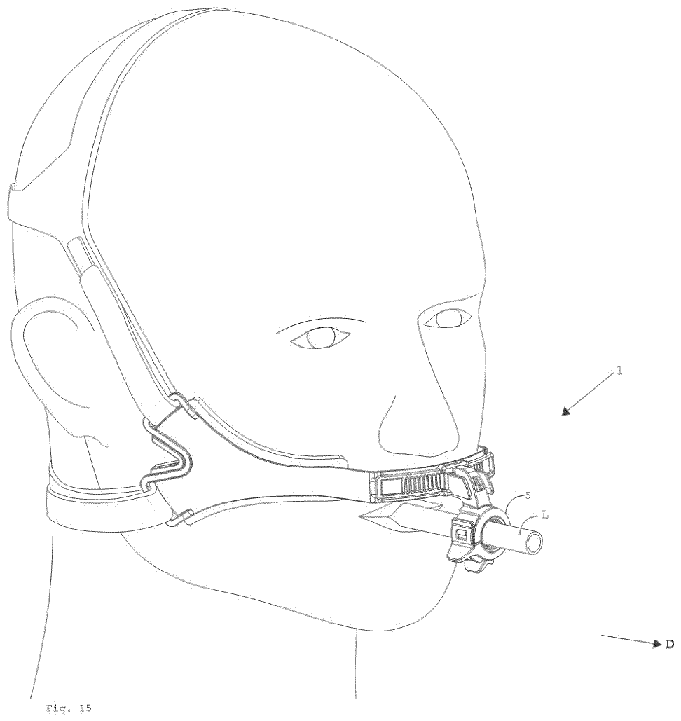

FIGS. 1-2 show a fixation device for fixing a medical line to a patient, in particular an endotracheal tube or to the line L as shown in FIG. 15. The fixation device 1 comprises a support member 3 supporting a retainer 5 for retaining the line. The retainer 5 defines an axis A and is adapted to fix the line L extending in axial direction, i.e. over or parallel to the axis A. As best seen in FIG. 15, the support member 3 is configured for mounting the fixation device 1 to the head of a patient, such that the retainer 5 is positioned in front of the mouth or nose of the patient to be intubated and the axis A extends towards the point of intubation (mouth or nose) in proximal-distal relation to the patient, as indicated with arrows P and D, respectively.

The shown support member 3 comprises a main member 7 which comprises facial rest portions, here cheek rests 9, and attachment portions 11 for one or more head bands, e.g. as shown in FIG. 15. The head bands may be elastic or non-elastic and they may be size adjustable, e.g. by doubling up of a band portion to form a loop and attachment of one end to an adjacent portion in a desired position using one or more displaceable buckles, a hook-and-loop type fastener (like Velcro® or similar) as indicated in FIG. 15, or other attachment technique like buttons, zippers, ratchet-portions (like tie-rips or similar). The shown cheek rests 9 are provided with cushioning skin contact pads 13, removably attached to the main member 7 with clamps 15 that are detachably fixable to the main member 7. The pads 13 may be adhesive but preferably they are non-adhesive. The materials of the contact pads 13 and clamps 15 may differ. The pads 13 may be flexible, or at least comprise a flexible material, e.g. a foam. The pads may comprise a coating, a laminate and/or another surface finish, e.g. a density gradient in a foam-material. The clamps 15 are generally solid, preferably resilient. The contact pads 13 and clamps 15 may be attached, preferably fixed, together from separate parts, e.g. by stitching, gluing, welding, etc., or they may be formed as a unitary object. A surface finish on one or both of a pad 13 and a clamp 15 may facilitate attachment of them together. The attachment may be substantially permanent or detachable.

Non-adhesive and/or exchangeable pads 13 may reduce or prevent skin damage, in particular in case of prolonged use. Also, different skin pad sizes and/or textures may be used. The main member 7 here is generally formed as a bridge, supporting a track 17 on the cheek rests 9 by struts 19. Thus, the track 17 may be kept separated from the face of the patient, in particular from the patient's lips and nose (cf. FIG. 15).

The track 17 extends lateral to the axis A (FIG. 1) and comprises rails 21 and a series of teeth 23.

In other embodiments, not shown, the retainer 5 may be supported on a differently formed support member for mounting on another patient body part, e.g. for fixing a catheter, a drain, a sensor line and/or an infusion line.

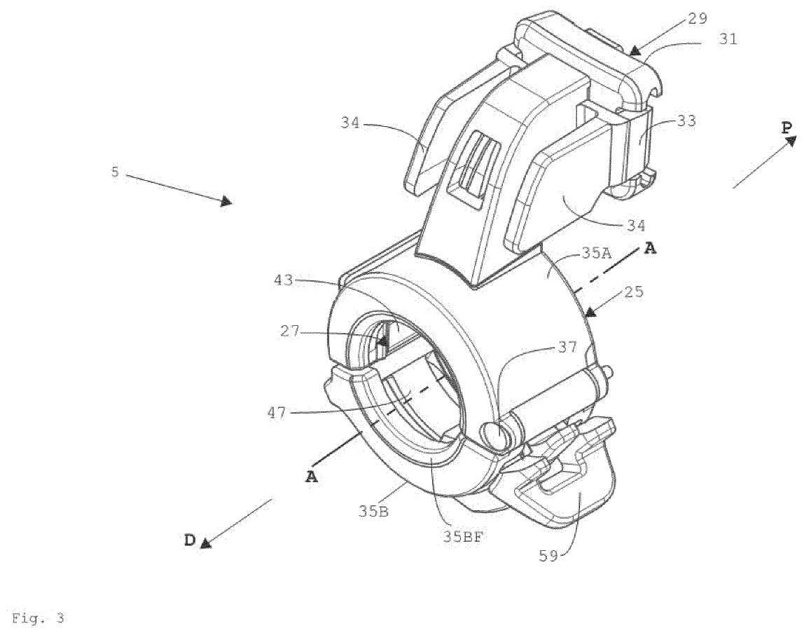

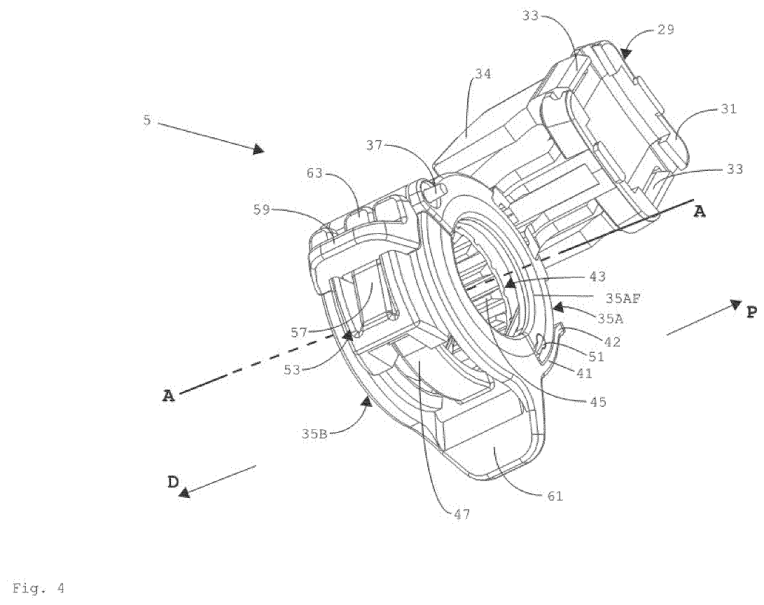

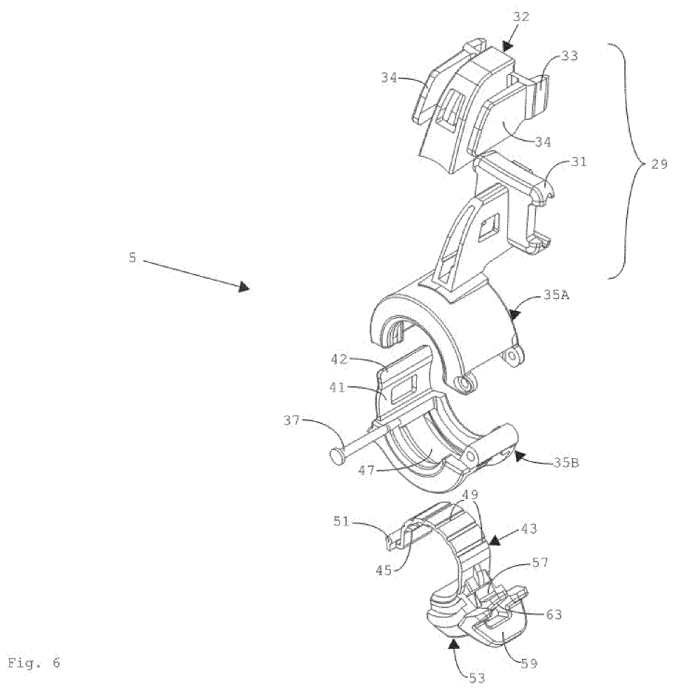

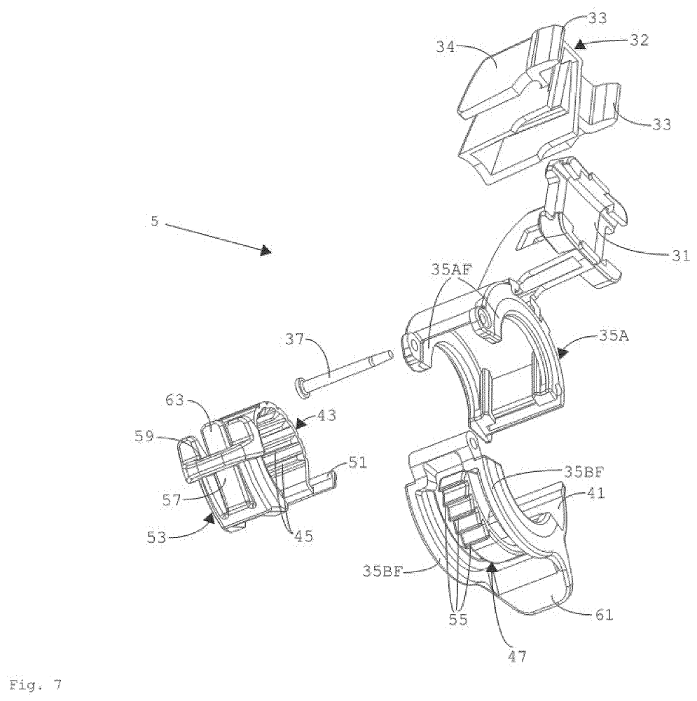

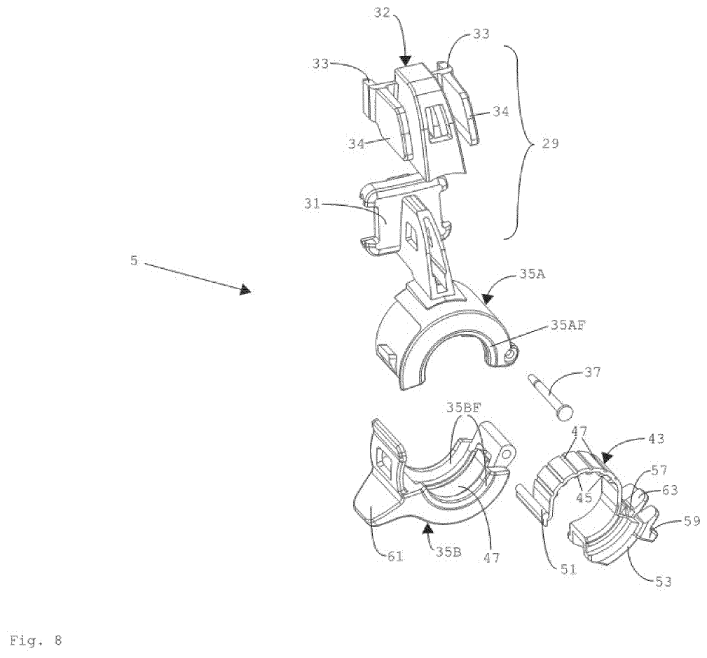

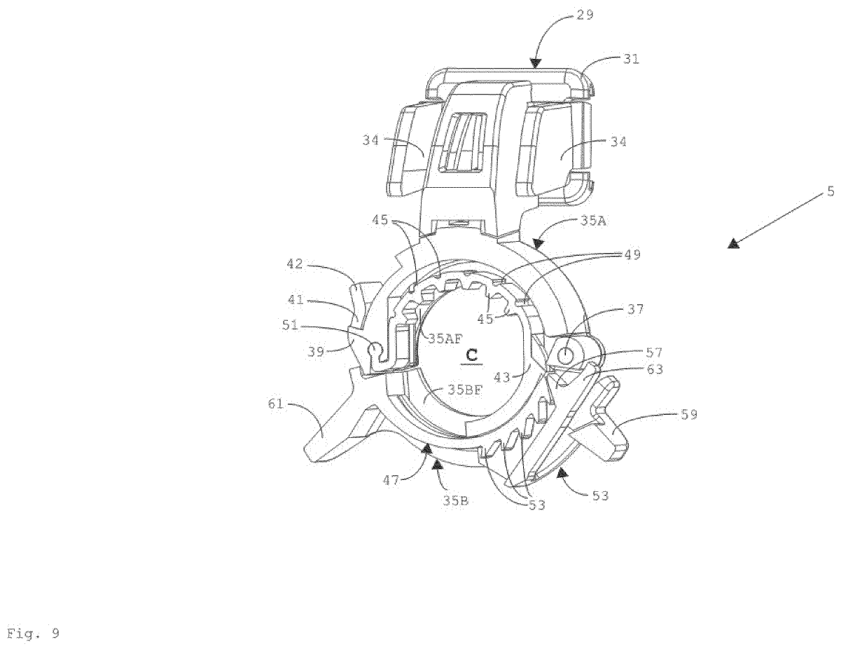

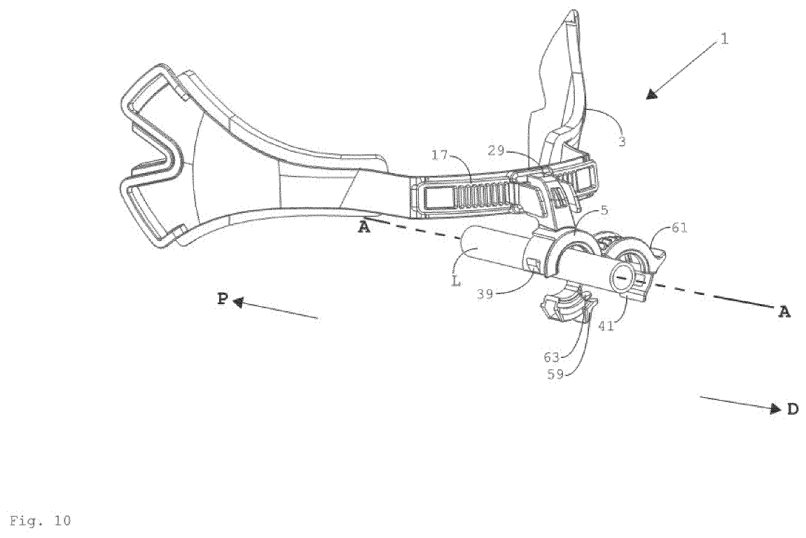

FIGS. 3-8 are various views of the retainer 5 separately. FIG. 9 is a cross section view. FIGS. 10-13 show use of the device (partially shown). Referring to FIGS. 3-9, the retainer 5 comprises an enclosure 25 for radially retaining the line in axial direction along the axis A, a clamp 27, and further a mount 29 for connecting the retainer 5 to the support member 3, in particular to the track 17 thereof.

In the shown embodiment the mount 29 comprises a connector 31 for slidably coupling the retainer 5 to the rails 21 of the track 17 and pawls 33 for engaging teeth 23 of the track 17. Here, the pawls 33 are provided on a separate cap 32 fitted to the connector 31, e.g. by snapping. Combining separate retainer parts (e.g. here connector 31 and cap 32) facilitates selecting different materials for the different retainer parts and/or simplifying a mold in case one or more parts of the retainer 5 are molded. The pawls 33 are movable relative to the connector 31 using tabs 34. Thus, when (the mount 29 of) the retainer 5 is operably coupled to the track 17 (see FIG. 1) the pawls 33 can be selectively made to engage/disengage teeth 23 of the track 17 so that the retainer 5 can be slid laterally along the track 17 or be fixed to the latter in several desired relative positions.

The shown enclosure 25 comprises two arms 35A, 35B formed as C-shaped parts hingedly connected to each other at respective tips, here with a pivot 37 which extends parallel to the axis A and offset from the latter, but other hinges, e.g. one or more film hinges, may be provided.

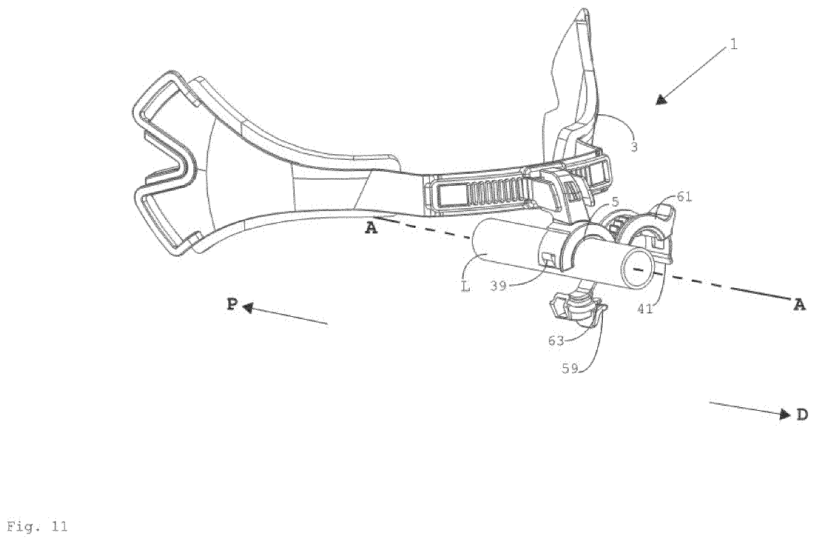

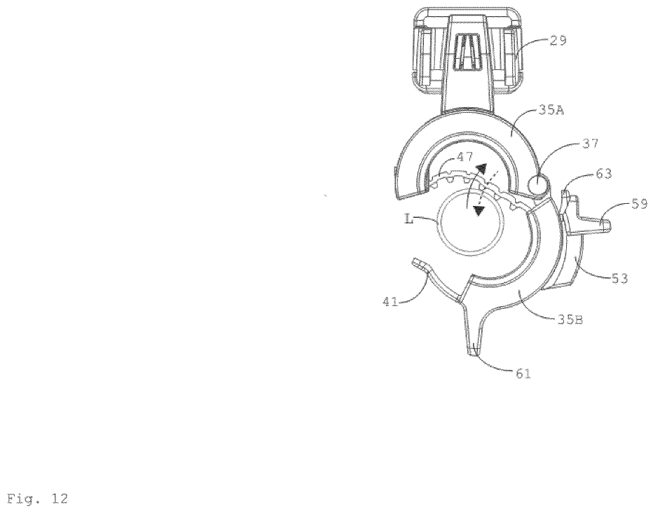

Due to the hinging connection of the arms 35A, 35B, the arms 35A, 35B are movable from a closed configuration, here a combined O-shape (FIGS. 3-5, 9) for radially retaining the line (FIG. 12), to an open configuration for receiving the line (FIGS. 10-11) in which open configuration, the arms 35A, 35B together may form a more-or-less U- or W-shape, depending on the amount of opening and the relative positions of the arms 35A, 35B. Here, the arms 35A, 35B each are substantially semi-circular but other shapes such as elliptical or rectangular may be used.

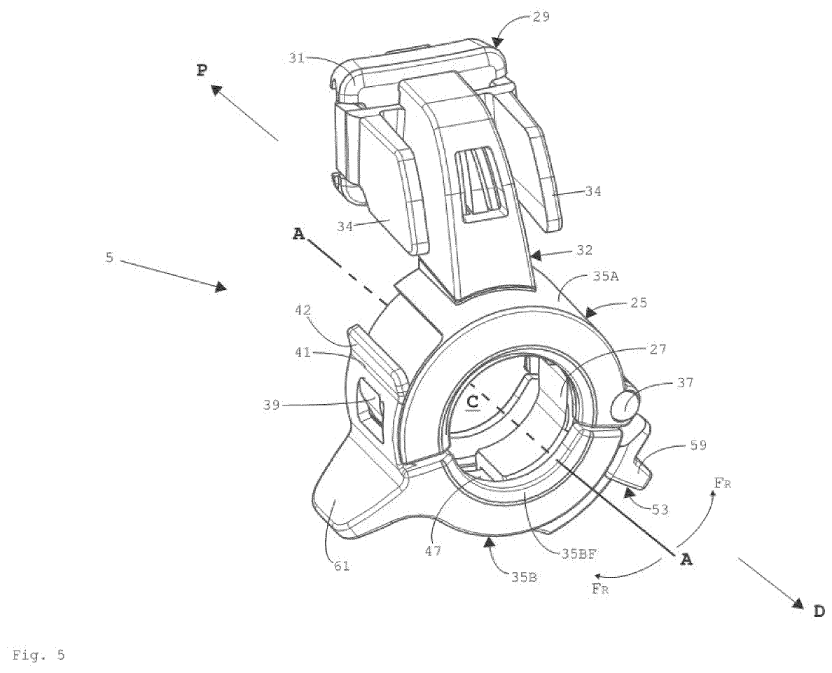

The enclosure 25 comprises a latch 39 on one arm 35A and a matching catch 41 on the other arm 35B which together form a snap lock for closing the enclosure 25 such that it forms a fully closed device encircling the axis A and providing an open channel C. Note that each arm 35A, 35B comprises, on axially opposite ends, radial inward extending flange portions 35AF, 35BF which are located in a common radial plane and provide rings around the axis A. The flange portions 35AF, 35BF may support the line on opposite sides of the clamp 27 (see below). Such opposite support, in particular if extending over more than 180 degrees around the axis as here, can absorb lateral forces, e.g. rotation forces F_{R} if the line and/or the device is pulled sideways (see FIG. 5), e.g. by movement of a patient's head so that the clamp 27 may be, at least largely, spared from such forces F_{R}, relaxing robustness requirements for the clamp 27.

Provision of an optional release to the lock (39+41), here in the form of a tab 42, facilitates opening of the enclosure 25, such that that can be reversibly, and preferably repeatedly, closed and opened. Note that other locks than a snap lock may be used.

The clamp 27 comprises a clamp member 43 which comprises radial inward protrusions 45. The clamp 27 further comprises a second clamp member 47.

The shown clamp member 43 is flexibly deformable, preferably resilient, which properties may be due to material choices and/or shaping of the clamp member 43, e.g. by recesses 49 reducing thickness variations by the protrusions 45.

In the shown embodiment, the clamp member 43 is attached on one side to one arm 35A, here being attached with a key 51 fit in a matching groove in the arm 35A and extending parallel to the axis A and offset from the latter. The clamp member 43 is flexible allowing movement and deformation with respect to the key 51 and the arm 35A. In another embodiment (not shown), instead of a key and groove connection, hinges, e.g. including a pivot and/or including one or more film hinges, may be provided. Also, or alternatively, the clamp member may be integral with the enclosure (not shown). An opposite side of the clamp member 43 is attached to a clamp connector 53 with is in turn attached to the second clamp member 47. The clamp member 43 extends, as shown, over more than 180 degrees around the axis A.

The second clamp member 47 is attached on one side to one arm 35B. Here, as an option, the second clamp member 47 is formed integral with the arm 35B so that the second clamp member 47 may be seen as a portion of the arm 35B. However, the second clamp member 47 may also be a separate part hingedly attached with a pivot or other hinge to the enclosure, like the clamp member 43 discussed above.

The second clamp member 47 may be, as intended here, be resiliently flexible. The clamp member 43 and the second clamp member 47 are adjustably couplable or coupled together via the clamp connector 53. Thus coupled, the clamp 27 is openable, here on an opposite side, by opening of the enclosure 25 so that a line to be clamped can be introduced into the clamp 27 from the open side; see FIGS. 10-11. In closed configuration of the enclosure 25 the (e.g. see FIGS. 1, 3-5, 9) the clamp 27 extends fully around the axis A.

Visible in FIG. 12, is that since the clamp member 43 and the second clamp member 47 are attached to different arms 35A, 35B of the enclosure 25, in an open configuration of the retainer 5, the clamp member 43 and the second clamp member 47 are deformed. Introducing a line L into the retainer 5 may cause that the line L engages the clamp member 43 and, on pressing the line into the retainer 5 further (see solid arrow in FIG. 12), urges the clamp member 43 (more) into the arm 35A, thus pulling both arms 35A, 35B towards a closed configuration. Thus, stretching or opening of the clamp 27 and causing deformation of the retainer 5 towards a holding and/or clamping configuration by insertion of the line L to be clamped itself, ease of use is increased and proper alignment of the line L to a position for retainment and clamping is assisted. Note that, in reverse, on opening of the retainer 5 holding a clamped line L, stretching the clamp member 43 assists ejecting and removing the line L from the retainer 5, as indicated in FIG. 12 with a dashed arrow.

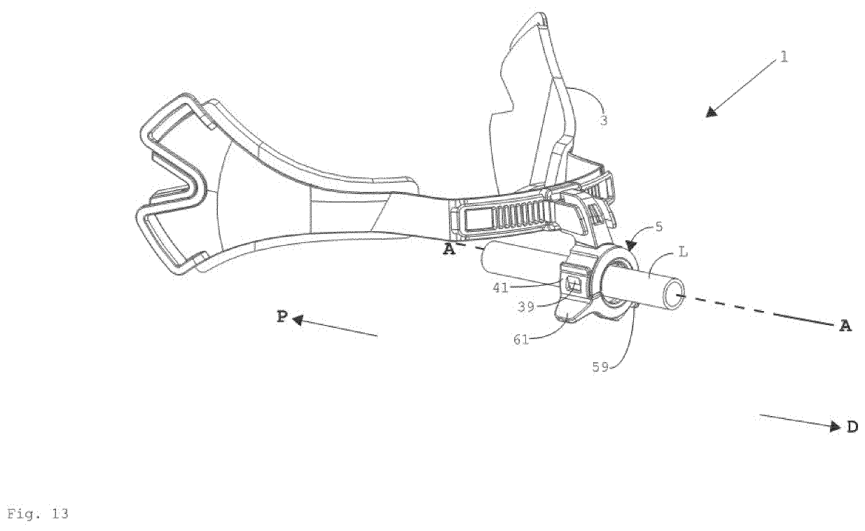

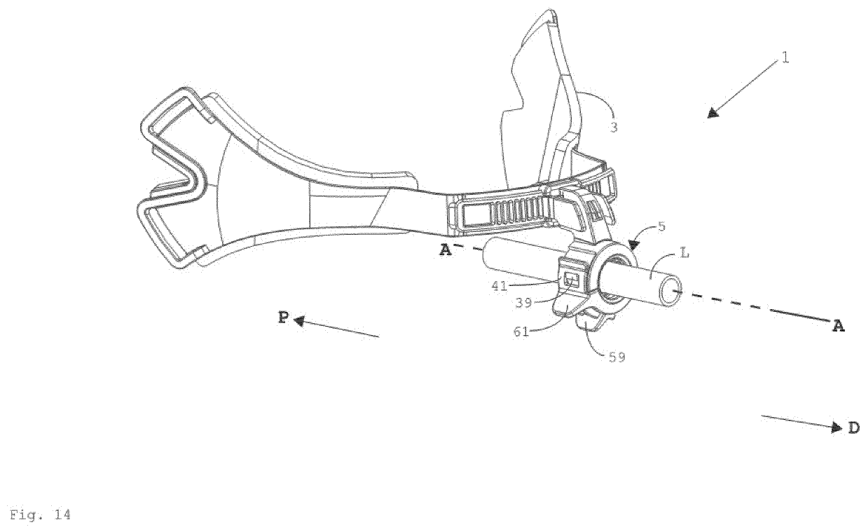

The connector 53 is movable over the second clamp member 47. The second clamp member 47 and the clamp connector 53 comprise, respectively teeth 55 and a pawl 57 forming a ratchet-and-pawl connection. By moving the clamp connector 53 and the second clamp member 47 relative to each other, in particular in closed configuration of (the enclosure 27 of) the retainer 5 and therewith of the clamp 27, the clamp 27 is configured to be tightened in circumferential direction around the axis A so that an opening of the clamp 27 accommodating the line is reduced. This is comparable to tightening a cable binder of the so-called “tie wrap”- or “tie rip”-types. Thus, a crimping force can be provided on the line L. For adjusting the relative position of the connector to the second clamp member 47 the connector 53 is optionally provided with a tab 59 and (an arm 35B of) the enclosure 25 is optionally provided with a tab 61; the tabs 59, 61 may be pulled together by pinching, which may e.g. be done by a thumb and forefinger of one hand, see also FIGS. 13 and 14.

For releasing the clamp 27 once it is tightened, the retainer 5 may be opened as a whole (here, by unlocking the latch 39 and catch 41 snap lock), or the pawl 57 may be withdrawn to disengage it from the teeth 55. For such release of the clamp 27 the pawl 57 may be provided with a tab 63.

The present retainer 5 can accommodate lines L of any diameter fitting inside the enclosure 25 when that is closed, and it can clamp lines of various smaller diameter than the maximum diameter, depending on, i.e., flexibility of the clamp members 43, 47 and relative sizes of the clamp connector 53.

The disclosure is not restricted to the above described embodiments which can be varied in a number of ways within the scope of the claims. For instance, the support member may have different shapes. The retainer may be larger or smaller. Plural retainers may be connected to one support member. Plural retainers for different lines may be connected to each other and be connected to a single support member as an assembly. The retainer may be fixed to a support member. The support member may not have a bridge portion but be configured for fully contacting a patient. Tabs may be made smaller or larger. The retainer may have a longer or shorter size in axial direction.

The head band may have another shape and/or be attached differently.

The device may be combined with a bite-block, known per se. The latter may be coupled to the support member and/or the retainer 5, e.g. by a coupling to the mount 29. The latter may e.g. be facilitated by adaptation of the cap 32.

Elements and aspects discussed for or in relation with a particular embodiment may be suitably combined with elements and aspects of other embodiments, unless explicitly stated otherwise.

1. A fixation device for a medical line to a patient, comprising
a support member for supporting a retainer on a patient, the retainer defining an axis and being adapted to retain the medical line extending in an axial direction,
wherein the retainer comprises
an enclosure for radially retaining the medical line and
a clamp,
wherein the clamp is configured to provide a crimping force around the axis for fixing the medical line in at least the axial direction relative to the enclosure.

2. The fixation device of claim 1, wherein the clamp is configured to provide a substantially axisymmetric clamping force distribution.

3. The fixation device of claim 1, wherein the clamp comprises a deformable member configured to be tightened in a circumferential direction around the axis.

4. The fixation device of claim 1, wherein the clamp is adjustable to accommodate and clamp different shapes and/or sizes of medical lines.

5. The fixation device of claim 1, wherein the clamp further comprises one or more indexing features and/or fixation features for establishing a size and/or a clamping force.

6. The fixation device of claim 1, wherein the retainer is openable and closable, and, when in an open state the retainer accepting the medical line to be clamped in a direction other than an axial direction, and in a closed state the retainer is configured for clamping the medical line.

7. The fixation device of claim 1, wherein the enclosure and/or the clamp extends over more than 180 degrees around the axis in an axial position.

8. The fixation device of claim 1, wherein the enclosure comprises plural parts hingedly connected for opening and closing the enclosure.

9. The fixation device of any preceding claim, wherein at least one of the enclosure and the clamp comprises a snap lock for closing.

10. The fixation device of claim 1, wherein the clamp is connected to plural parts of the enclosure and is openable or reopenable and/or closable or reclosable together with the enclosure.

11. The fixation device of claim 1, further comprising a clamp member, which is attached to or configured to be attached to two enclosure parts that are movable with respect to each other for opening and/or closing the enclosure.

12. The fixation device of claim 1, wherein the clamp is accommodated at least partly within the enclosure.

13. The fixation device of claim 1, wherein the retainer is movable and adjustable relative to the support member to plural positions on the support member.

14. The fixation device of claim 1, wherein the support member comprises a lateral track and the retainer is movable along the lateral track.

15. The fixation device of claim 1, wherein the support member is configured for head-mounting of the fixation device, said fixation device further comprising one or more facial rest portions, and/or
wherein the support member comprises a bridge for maintaining one or more portions of the support member free from skin contact.

16. The fixation device of claim 1, wherein the fixation device is an endotracheal tube.

17. The fixation device of claim 1, wherein the crimping force also fixes the medical line in a radial direction relative to the enclosure.

18. The fixation device of claim 7, wherein the enclosure and/or the clamp fully surrounds the axis in the axial position.

19. The fixation device of claim 8, wherein the plural parts are two or more C-shaped parts hingedly connected at adjacent respective tip ends.

20. The fixation device of claim 15, wherein the bridge comprises or supports the lateral track of claim 14.
